(19) United States
(12) Patent Application Publication
(10) Pub. No.: US 2019/0387963 A1
(43) Pub. Date: Dec. 26, 2019

(54) IMAGING APPARATUS AND ENDOSCOPE

(71) Applicant: OLYMPUS CORPORATION, Tokyo (JP)

(72) Inventors: Keigo MATSUO, Tokyo (JP); Hisashi ODE, Tokyo (JP); Toshiro OKAMURA, Tokyo (JP); Atsushi DOI, Tokyo (JP); Satoshi WATANABE, Tokyo (JP)

(73) Assignee: OLYMPUS CORPORATION, Tokyo (JP)

(21) Appl. No.: 16/561,816

(22) Filed: Sep. 5, 2019

Related U.S. Application Data
(63) Continuation of application No. PCT/JP2017/008689, filed on Mar. 6, 2017.

(51) Int. Cl. A61B 1/05; A61B 1/06; A61B 1/00

(52) U.S. Cl. CPC A61B 1/051; A61B 1/06; H04N 2005/2255; A61B 1/00096; A61B 1/00009; A61B 1/0019

(57) ABSTRACT

A lens system is configured to form an image of an object on an imaging surface of an image sensor. A branching optical system is configured such that an image-forming light flux in the lens system is split and propagated into a plurality of optical paths having different optical path lengths and such that the principal rays of a plurality of image-forming light fluxes obtained after the splitting are incident on a predetermined region of the image sensor.

### BACKGROUND OF THE INVENTION

### 1. Field of the Invention

The present invention relates to an imaging apparatus.

### 2. Description of the Related Art

Depth of field is known as one of the characteristics of a lens system. The term depth of field refers to a range where a focusing portion appears to be in focus from front to back when an image is captured through a lens system.

In art photography and the video production field, a shallow depth of field is actively used for expression. On the other hand, in some applications, an image having a deep depth of field (a large range that appears to be in focus) may be required (the image is also referred to as a pan-focus image). For example, microscopes and endoscopes are examples of those for which a deep depth of field is required. Even in general photography, for example, when imaging a person and a landscape, there is a need to focus on both of them.

In order to increase the depth of field, following approaches can be taken: (i) increase an aperture value (f-number); (ii) reduce the focal length of the lens; and (iii) increase the working distance from the camera to an object. Since (ii) and (iii) are often limited depending on the application, the simplest approach is to increase the aperture value. However, if the aperture value is increased, the amount of light taken into an image sensor decreases. Thus, in order to capture an image of the same brightness, it is necessary to increase the sensitivity of the image sensor or increase the exposure time. When the sensitivity is increased, the image quality is lowered. When the exposure time is increased, image capturing is likely to be affected by camera shake and motion blur. The same applies to moving images, and when the exposure time is increased, it is difficult to capture images at a desired frame rate.

Several techniques have been suggested to increase the depth of field without increasing the aperture value (for example, Sujit Kuthirummal, Hajime Nagahara, Changyin Zhou, and Shree K. Nayar, “Focal Sweep Videography with Deformable Optics”, IEEE Transactions on Pattern Recognition and Machine Intelligence, Vol. 33, No. 1, pp. 58-71, 2011.01, hereinafter referred to as Non-Patent Document 1). FIG. 1 is a diagram showing the outline of the prior art for increasing the depth of field. FIG. 1 shows a state in which an object point M is in focus and the object point M forms an image on an imaging surface of an image sensor 12 through a lens system (image forming optical system) 10. When the image sensor 12 is shifted back and forth, a captured image becomes blurred.

In the technique according to Non-Patent Document 1, the image sensor 12 is shifted along the optical axis during exposure (focal sweep photographing). An image obtained as a result is integration of images at respective image capturing positions.

Capturing the image of a point light source by focal sweep is now considered. When a point light source is present in a range conjugate with the shift range of the image sensor 12, an image captured by focal sweep is referred to as an integrated point spread function (hereinafter, IPSF). This IPSF is constant independently of the position of the point light source. Using this property, an image with an expanded depth of field can be obtained by the deconvolution, using an IPSF, of an object image captured by focal sweep.

Non-Patent Document 2 (D. Miau, O. Cossairt and S. K. Nayar, “Focal Sweep Videography with Deformable Optics”, IEEE International Conference on Computational Photography (ICCP), pp. 1-8, April 2013) discloses a technique for acquiring a similar image by driving a lens system instead of shifting an image sensor.

In the techniques disclosed in Non-Patent Documents 1 and 2, it is necessary to drive an image sensor or a lens system during exposure, which results in the complication of control and/or devices.

### SUMMARY OF THE INVENTION

In this background, one of exemplary purposes of an embodiment of the present invention is to provide an imaging apparatus capable of acquiring a focal sweep image without driving an image sensor or a lens system during exposure.

One embodiment of the present invention relates to an imaging apparatus. The imaging apparatus includes: an image sensor; a lens system configured to form an image of an object on an imaging surface of the image sensor; and a branching optical system configured such that an image-forming light flux in the lens system is split and propagated into a plurality of optical paths having different optical path lengths and such that the principal rays of a plurality of image-forming light fluxes obtained after the splitting are incident on a predetermined region of the image sensor.

One embodiment of the present invention relates to an imaging apparatus. The imaging apparatus includes: an image sensor; a lens system configured to form an image of an object on an imaging surface of the image sensor; and a branching optical system configured such that an image-forming light flux in the lens system is split and propagated and such that image-forming points of respective imaging-forming light fluxes after the splitting are located at different distances from the imaging surface.

Optional combinations of the aforementioned constituting elements, and implementations of the invention in the form of methods, apparatuses, and systems may also be practiced as additional modes of the present invention.

### BRIEF DESCRIPTION OF THE DRAWINGS

Embodiments will now be described, by way of example only, with reference to the accompanying drawings which are meant to be exemplary, not limiting, and wherein like elements are numbered alike in several Figures, in which:

FIG. 1 is a diagram showing the outline of the prior art for increasing the depth of field.

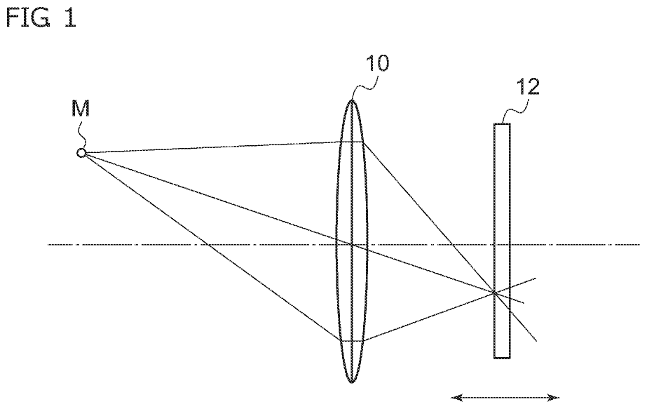

FIG. 2 is a diagram showing the basic configuration of an imaging system provided with an imaging apparatus according to an embodiment.

FIG. 3 is a diagram schematically showing the imaging apparatus according to the embodiment.

FIGS. 4A to 4C are diagrams showing images of a point light source captured by the imaging apparatus of FIG. 2.

FIG. 5 is a diagram showing an imaging apparatus according to a first exemplary embodiment.

FIG. 6 is a diagram showing an imaging apparatus according to a second exemplary embodiment.

FIG. 7 is a diagram showing an imaging apparatus according to a third exemplary embodiment.

FIG. 8 is a diagram showing an imaging apparatus according to a fourth exemplary embodiment.

FIG. 9 is a diagram showing an imaging apparatus according to a fifth exemplary embodiment.

FIG. 10 is a diagram showing a distal end portion of an endoscope.

### DETAILED DESCRIPTION OF THE INVENTION

The invention will now be described based on preferred embodiments which do not intend to limit the scope of the present invention but exemplify the invention. All of the features and the combinations thereof described in the embodiment are not necessarily essential to the invention.

First, the outline of some embodiments according to the present invention will be described. An imaging apparatus according to an embodiment of the present invention includes an image sensor, a lens system configured to form an image of an object on an imaging surface of the image sensor, and a branching optical system configured such that an image-forming light flux in the lens system is split and propagated into a plurality of optical paths having different optical path lengths and such that the principal rays of a plurality of image-forming light fluxes obtained after the splitting are incident on a predetermined region of the image sensor.

The image sensor allows for the simultaneous image capturing of the image-forming light fluxes propagated through the plurality of optical paths having different optical path lengths, and this is equivalent to exposure performed while sweeping the focus. According to this embodiment, sweeping of the image sensor during exposure and driving of the lens system are not necessary.

The branching optical system may include a Michelson interferometer type optical system that includes a beam splitter and two mirrors. By setting an optical path difference between the two arms, two optical paths can be formed.

The beam splitter is a polarization beam splitter, and the system may further include a quarter-wave plate provided between each of the two mirrors and the beam splitter. This can reduce the loss of light.

The branching optical system may further include at least one parallel plate inserted between at least one of the two mirrors and the beam splitter, and the parallel plate may be configured to partially reflect light at the front and back surfaces thereof. Thus, an arm (optical path) into which the parallel plate is inserted can be further split into a plurality of optical paths.

The branching optical system may include at least one parallel plate provided on the object side of the lens system or on the image sensor side of the lens system, and the parallel plate may be configured to partially reflect light at the front and back surfaces thereof.

A description will be given of the present invention with reference to the drawings based on preferred embodiments. The same or equivalent constituting elements, members, and processes illustrated in each drawing shall be denoted by the same reference numerals, and duplicative explanations will be omitted appropriately. Further, the embodiments do not limit the invention and are shown for illustrative purposes, and all the features described in the embodiments and combinations thereof are not necessarily essential to the invention.

Further, the dimensions (thickness, length, width, etc.) of each member described in the drawings may be scaled up or down as appropriate to facilitate understanding. Furthermore, the dimensions of a plurality of members do not necessarily represent the magnitude relationship between them, and even if one member A is drawn thicker than another member B in the drawing, the member A can be thinner than the member B.

The vertical and horizontal axes of the graphs referred to in the present specification are scaled up or down as appropriate to facilitate understanding, and the graphs and waveforms shown are also simplified for easy understanding or are exaggerated or emphasized.

### Overview and Principle

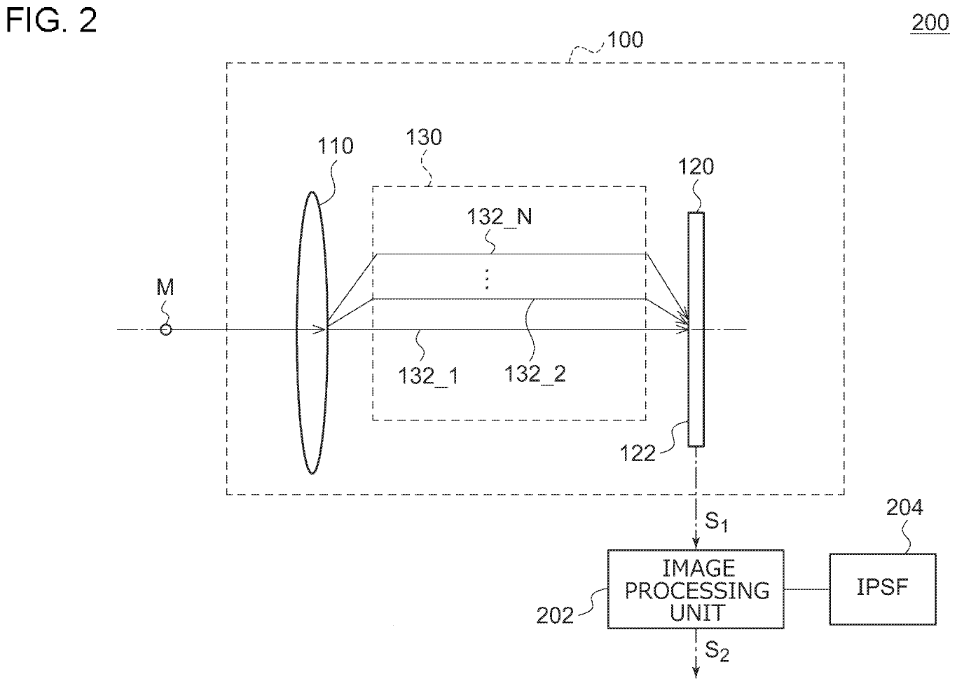

FIG. 2 is a diagram showing the basic configuration of an imaging system 200 provided with an imaging apparatus 100 according to an embodiment. The imaging apparatus 100 includes a lens system 110, an image sensor 120, and a branching optical system 130. In the present specification, light rays are indicated by solid lines, and signal lines and data lines are indicated by alternate long and short dash lines.

The image sensor 120 is a charge coupled device (CCD), a complementary metal oxide semiconductor (CMOS) sensor, an organic light sensor, or the like and has a plurality of pixels arranged in a matrix. The lens system 110 is an image forming optical system configured to form an image of an object on an imaging surface 122 of the image sensor 120. In the present specification, the lens system 110 is represented by a single lens; however, the configuration thereof is not particularly limited, and various known techniques can be adopted.

The branching optical system 130 splits and propagates an image-forming light flux in the lens system 110 into a plurality of optical paths 132_1 to 132_N having different optical path lengths. The number N of the optical paths 132 is not particularly limited. The branching optical system 130 is configured such that the principal rays of a plurality of image-forming light fluxes obtained after the splitting are incident on a predetermined region (substantially the same part) of the image sensor 120. The “predetermined region” means a part that corresponds to the image-forming point for each object point. The predetermined region preferably corresponds to one point and may be, for example, a range that can be considered to be in the same pixel of the image sensor. Alternatively, when a certain degree of image quality deterioration is allowed, the incident position of each principal ray may be shifted, for example, the predetermined region needs to fit into a few adjacent pixels, and the predetermined area thus becomes wider.

For easy understanding, FIG. 2 shows only the point light source M and, for a ray of light, shows only a principal ray. Also, the optical paths 132 shown in FIG. 2 are shown conceptually, and the shapes thereof merely indicate that the optical path lengths thereof are different. In the following explanation, it is assumed that the optical path length of each optical path is understood as the propagation distance of the principal ray of the imaging-forming light flux propagating therethrough.

The image processing unit 202 processes image data S₁ obtained by the imaging apparatus 100 so as to restore an image having a deep depth of field. The process performed by the image processing unit 202 may be the same as that of the known techniques and therefore will be briefly described. A memory 204 stores data including the IPSF of the lens system 110 and the branching optical system 130. The image data S₁ acquired by the imaging apparatus 100 is deconvoluted using the IPSF so as to generate an image S₂ in which the depth of field is expanded. Although the image processing unit 202 and the memory 204 are shown outside the imaging apparatus 100, these may be part of the imaging apparatus 100 as a matter of course.

The above is the basic configuration of the imaging apparatus 100. Subsequently, the operation thereof will be described. Focusing on a plurality of imaging-forming light fluxes propagating in the plurality of optical paths 132 of the branching optical system 130, although a light flux that has propagated through one optical path 132 can form an image on the imaging surface of the image sensor 120, light fluxes that have propagated the remaining optical paths 132 do not form an image on the imaging surface 122 of the image sensor 120 since the light fluxes are defocused by the branching optical system 130, and thus form a blurred image. The image sensor 120 captures a plurality of superimposed images formed by light propagated through the plurality of optical paths 132_1 to 132_N. This is equivalent to shifting the image sensor 120 at a plurality, N, of positions during exposure or equivalent to changing the focus position of the lens system 110 during exposure.

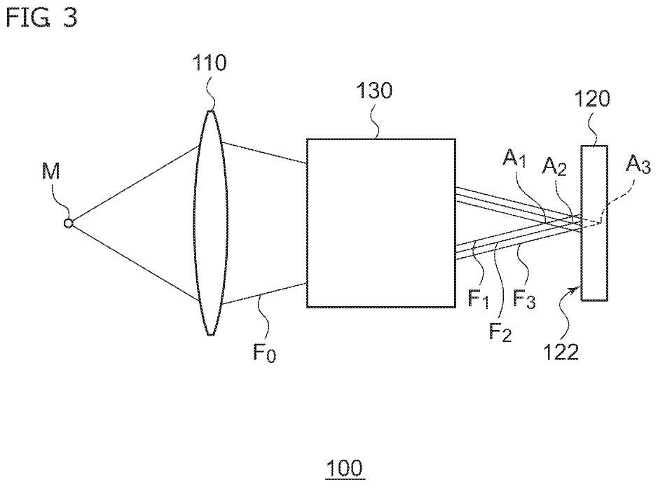

FIG. 3 is a diagram schematically showing the function of the branching optical system 130 of FIG. 2. As described above, the lens system 110 is configured to form an image of an object on the imaging surface 122 of the image sensor 120. The branching optical system 130 is configured such that an image-forming light flux F₀ in the lens system 110 is split and propagated and such that image-forming points A₁ to A₃ of respective imaging-forming light fluxes F₁ to F₃ after the splitting are located at different distances from the imaging surface 122.

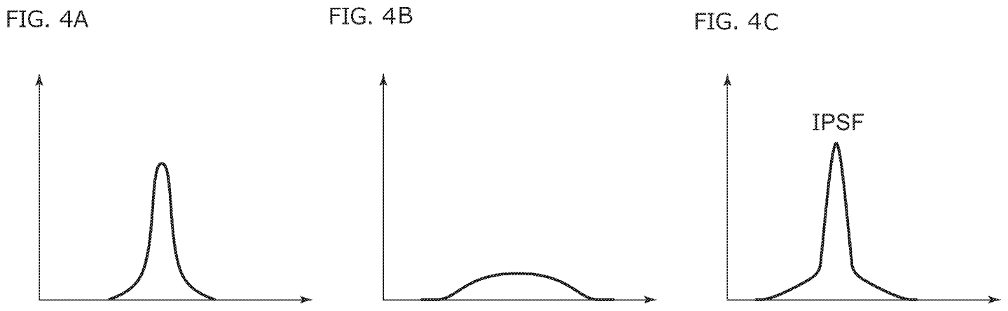

FIGS. 4A to 4C are diagrams showing images of a point light source captured by the imaging apparatus 100 of FIG. 2. Although the images are actually two-dimensional images, the images are shown as one-dimensional images in the figures where the horizontal axis represents the position and the vertical axis represents the intensity. In the figures, the number N of optical paths is two. FIGS. 4A and 4B show images formed on the image sensor 120 by image-forming light fluxes propagated through the first optical path 132_1 and the second optical path 132_2. For example, the image in FIG. 4A represents a point spread function (PSF) of an optical system including the first optical path 132_1 and the lens system 110. FIG. 4C represents an image obtained by superposing the PSFs of FIGS. 4A and 4B, which corresponds to an integrated point spread function (IPSF).

The IPSF shown in FIG. 4C can be acquired by calculation or by actually capturing the image of a point light source. What is stored in the memory of the image processing unit 202 is nothing but this IPSF.

The actual object can be understood as the aggregate of point light sources that are distributed three-dimensionally. An image S₁ captured by the imaging apparatus 100 is an image obtained by capturing an object while sweeping the focus during exposure.

The above is the operation of the imaging apparatus 100. According to this imaging apparatus 100, a focal sweep image can be acquired without shifting the image sensor 120 during exposure or driving the lens system 110.

Since sweeping of the image sensor during exposure and driving of the lens system are not necessary, the device can be simplified. This will contribute to the downsizing and cost reduction of the imaging apparatus 100. In addition, although timing control over the exposure and the sweeping has been required conventionally, such a complicated process is not necessary in the imaging apparatus 100.

In addition, since the process of the sweeping and the lens driving is unnecessary and a focal sweep image can be acquired by one shot, the exposure time can be shortened. This is advantageous for applications with restrictions on exposure time, such as flash photography and high frame rate moving image photography.

Conventionally, when the object or camera moves during mechanical focal sweeping, motion blur and/or camera shake cause deterioration in the image quality. However, according to the imaging apparatus 100, since the exposure time is short, the deterioration in the image quality due to motion blur and/or camera shake can be suppressed.

The present invention can be understood from FIG. 2 and FIG. 3 or extends to various devices and methods derived from the above explanation and is not limited to a specific configuration. Hereinafter, in order not to narrow the scope of the present invention but to help the understanding of the nature of the invention and the circuit operation and to clarify them, more specific exemplary embodiments and exemplary variations will be described.

### FIRST EXEMPLARY EMBODIMENT

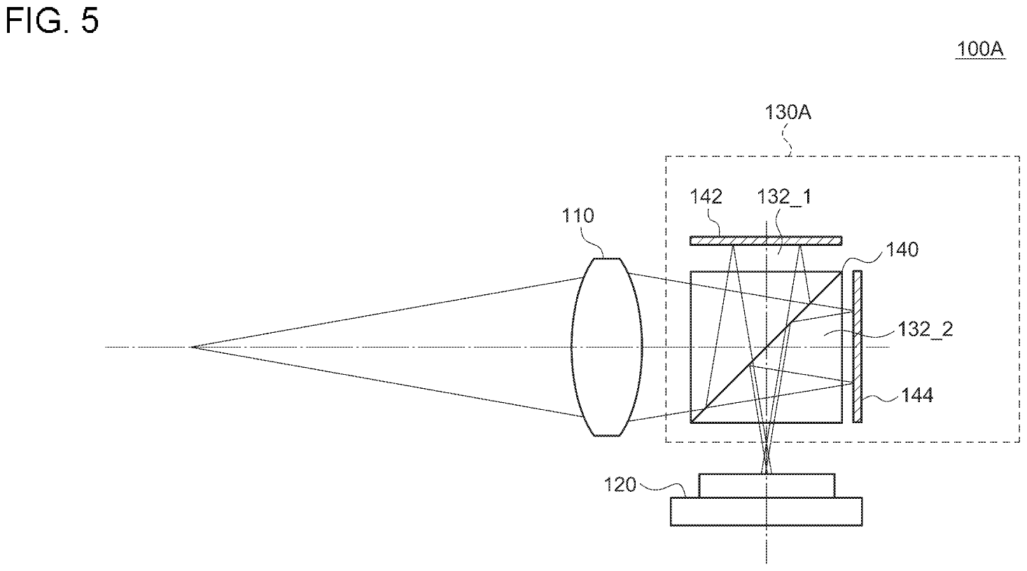

FIG. 5 is a diagram showing an imaging apparatus 100A according to the first exemplary embodiment. The branching optical system 130A is an optical system similar to a so-called Michelson interferometer and includes a beam splitter 140, a first mirror 142, and a second mirror 144. The beam splitter 140 splits the light transmitted through the lens system 110 into a reflection path (first arm) and a transmission path (second arm). The split beam of light in the first arm is reflected by the first mirror 142 and transmitted through the beam splitter 140 and becomes incident on the image sensor 120. The optical path including the first arm corresponds to the first optical path 132_1 of FIG. 2. The split beam of light in the second arm is reflected by the second mirror 144, reflected again by the beam splitter 140, and becomes incident on the image sensor 120. The optical path including the second arm corresponds to the second optical path 132_2 of FIG. 2. The distance (arm length) from the beam splitter 140 to the first mirror 142 and the distance from the beam splitter 140 to the second mirror 144 are different.

The above is the configuration of the branching optical system 130A according to the first exemplary embodiment. According to this branching optical system 130A, an image-forming light flux can be split into two optical paths having different optical path lengths. As a result, the imaging apparatus 100A can capture an image with two switched focuses in one shot. The position of the focus can be set according to the respective positions of the two mirrors 142 and 144.

Although the optical path length on the first optical path 132_1 side is longer in FIG. 5, the optical path length is not limited thereto. The optical path length of the second optical path 132_2 may be designed to be longer by moving the second mirror 144 away from the beam splitter 140 compared to the first mirror 142.

The optical path lengths may be made different by changing the distance from the beam splitter 140 to the first mirror 142 and the distance from the beam splitter 140 to the second mirror 144 to be equal and inserting a transparent material with a high refractive index in one of the arms.

### SECOND EXEMPLARY EMBODIMENT

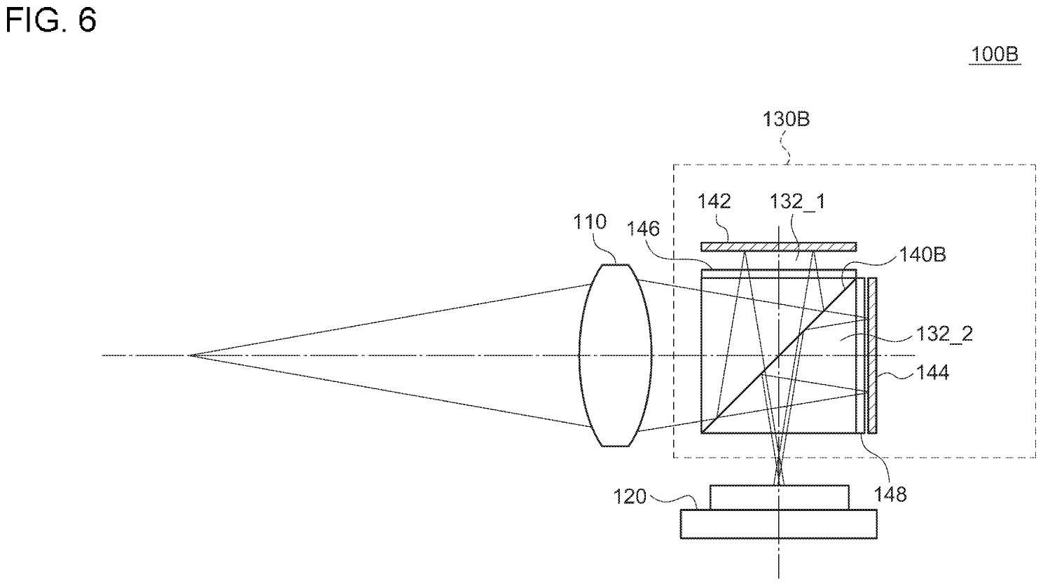

FIG. 6 is a diagram showing an imaging apparatus 100B according to the second exemplary embodiment. A branching optical system 130B also has an optical system similar to the Michelson interferometer. A beam splitter 140B in the branching optical system 130B is a polarization beam splitter. The branching optical system 130B includes a first λ/4 plate 146 and a second λ/4 plate 148 in addition to the branching optical system 130A. The first λ/4 plate 146 is inserted between the beam splitter 140B and the first mirror 142, and the second λ/4 plate 148 is inserted between the beam splitter 140B and the second mirror 144.

The above is the configuration of the branching optical system 130B. The beam splitter 140B reflects an S-polarization component contained in an image-forming light flux before the splitting and transmits a P-polarization component. The S-polarization component that has been reflected is transmitted through the first λ/4 plate 146, becomes a P-polarization component, and becomes incident on the beam splitter 140B again. As a result, the component is not reflected when the component becomes incident again, and all the component becomes incident on the image sensor 120. On the other hand, the P-polarization component that has been transmitted is transmitted through the second λ/4 plate 148, becomes an S-polarization component, and becomes incident on the beam splitter 140B again. As a result, the component is all reflected when the component becomes incident again, and becomes incident on the image sensor 120. As described above, according to the branching optical system 130B of FIG. 6, the loss of light can be reduced.

### THIRD EXEMPLARY EMBODIMENT

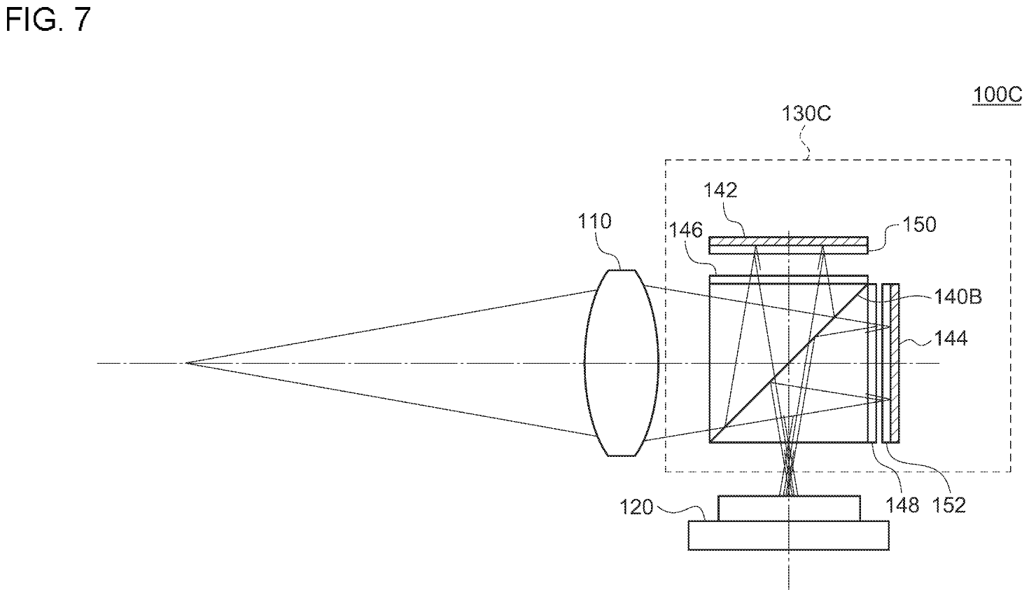

FIG. 7 is a diagram showing an imaging apparatus 100C according to the third exemplary embodiment. The branching optical system 130C further includes a first parallel plate 150 and a second parallel plate 152 in addition to the branching optical system 130B of FIG. 6. The parallel plates 150 and 152 are each configured to partially reflect light on the front and back surfaces thereof. In this exemplary embodiment, the back surface of the parallel plate 150 is the same as the front surface of the first mirror 142, and the back surface of the parallel plate 152 is the same as the front surface of the second mirror 144. For example, as the parallel plates 150 and 152, those with a surface coated with a transparent material such as glass can be used.

The above is the configuration of the imaging apparatus 100C. Focusing on the arm on the reflection side of the beam splitter 140B, light reflected by the beam splitter 140B is split into an optical path where the reflection occurs on the front surface of the first parallel plate 150 and an optical path where the reflection occurs on the back surface (first mirror 142) of the first parallel plate 150. Focusing on the arm on the transmission side of the beam splitter 140B, light transmitted through the beam splitter 140B is split into an optical path where the reflection occurs on the front surface of the second parallel plate 152 and an optical path where the reflection occurs on the back surface (second mirror 144) of the second parallel plate 152.

According to this branching optical system 130C, an image-forming light flux can be split into four optical paths having different optical path lengths. As a result, the imaging apparatus 100C can capture an image with four switched focuses in one shot. The optical path length difference of the plurality of optical paths can be controlled by the respective thicknesses of the parallel plates 150 and 152 and the respective positions of the two mirrors.

### FOURTH EXEMPLARY EMBODIMENT

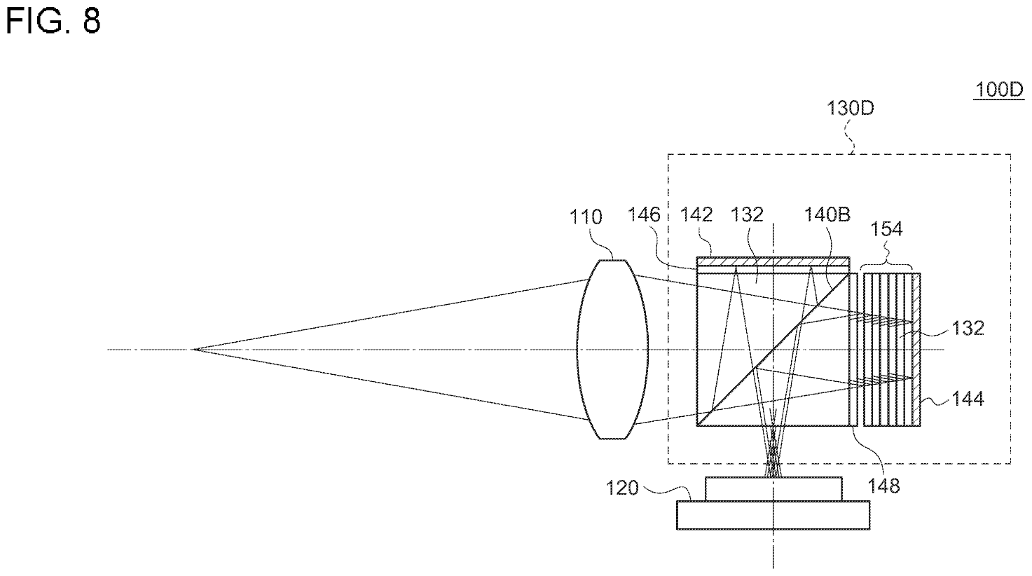

FIG. 8 is a diagram showing an imaging apparatus 100D according to the fourth exemplary embodiment. The branching optical system 130D further includes a parallel plate laminate structure 154 in addition to the branching optical system 130B of FIG. 6. The parallel plate laminate structure 154 is formed by stacking a plurality, N, of parallel plates, and each parallel plate is configured to partially reflect light on the front and back surfaces thereof.

The above is the configuration of the imaging apparatus 100D. Focusing on the arm on the transmission side of the beam splitter 140B, light transmitted through the beam splitter 140B is reflected at each layer of the parallel plate laminate structure 154. As a result, N+1 optical paths are formed in the transmission side arm. Since one optical path exists in the arm on the reflection side of the beam splitter 140B, according to the branching optical system 130D, an image-forming light flux can be split into N+2 optical paths having different optical path lengths, and the imaging apparatus 100D can capture an image with N+2 switched focuses in one shot. By increasing the number N of the layers, an image can be acquired that is close to the image obtained when the focus is swept continuously.

### FIFTH EXEMPLARY EMBODIMENT

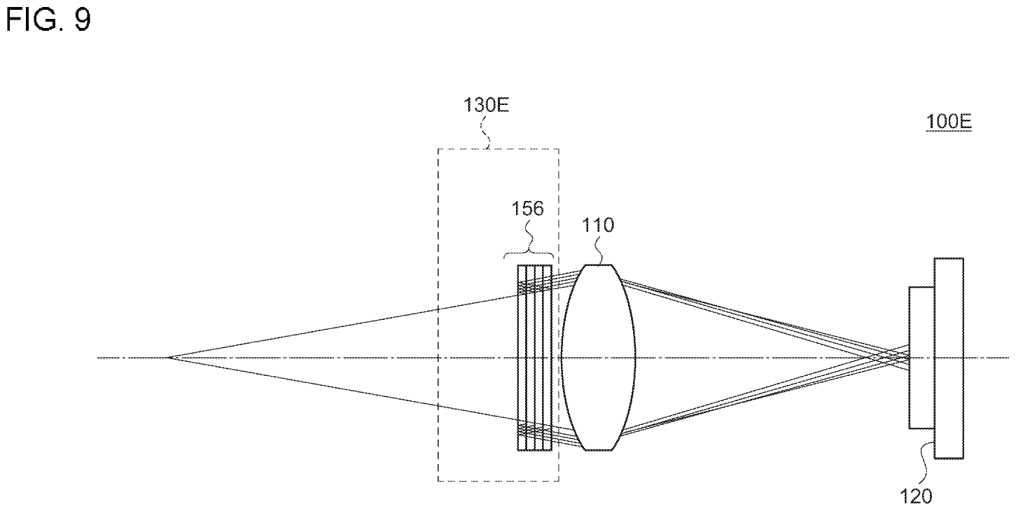

FIG. 9 is a diagram showing an imaging apparatus 100E according to the fifth exemplary embodiment. A branching optical system 130E includes at least one parallel plate 156 provided closer to the object than the lens system 110. In this exemplary embodiment, the parallel plate 156 has a laminate structure in which a plurality of layers are laminated.

The above is the configuration of the imaging apparatus 100E. An image-forming light flux is reflected on each layer of a parallel plate laminate structure 156 and is split and propagated to N+1 optical paths having different optical path lengths. The imaging apparatus 100E can capture an image with N+1 switched focuses in one shot.

In FIG. 9, the branching optical system 130E may be inserted on the image sensor 120 side of the lens system 110 or may be inserted on both sides.

The branching optical systems 130A to 130E according to the first to fifth exemplary embodiments described above can also be understood as follows. The branching optical systems include: an optical path branching surface that splits an image-forming light flux before the splitting into a first optical path and a second optical path; and at least one reflective surface arranged such that light propagating through the first optical path and light propagating through the second optical path become incident on the image sensor.

In the first and second exemplary embodiments, the surface of the beam splitter 140 corresponds to the optical path branching surface, and the first mirror 142 and the second mirror 144 correspond to the reflective surface.

In the third and fourth exemplary embodiments, the front surface of the beam splitter 140B and the front surfaces (back surfaces) of the parallel plates 150 and 152 correspond to the optical path branching surface. Further, the first mirror 142, the second mirror 144, and the front surfaces (back surfaces) of the parallel plates 150 and 152 correspond to the reflective surface.

In the fifth exemplary embodiment, the surface of each layer of the parallel plate laminate structure 156 is an optical path branching surface and corresponds to the reflective surface.

### Application

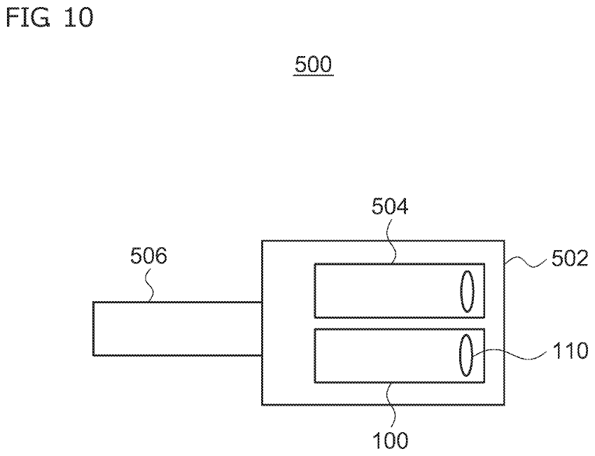

An imaging apparatus 100 can be used for an endoscope. FIG. 10 is a diagram showing a distal end portion 502 of an endoscope 500. The distal end portion 502 is attached to the distal end of a flexible insertion portion 506. The imaging apparatus 100 described above is housed in the case of the distal end portion 502 together with an illumination device 504. In the case of an endoscope 500 for three-dimensional shape measurement, the illumination device 504 may be an interference fringe projector. Inside the insertion portion 506, an optical fiber for supplying light from a light source to the illumination device 504, various power supplies, and a signal line for transmitting captured image data are inserted.

Endoscopes are required to have pan-focus images, and miniaturization of the imaging apparatus 100 is required at the same time. The imaging apparatus 100 according to the embodiments can satisfy such requirement of endoscopes at a high level.

The application of the imaging apparatus 100 is not limited to an endoscope and may be used for a microscope. Alternatively, the imaging apparatus 100 can be used for digital cameras and digital video cameras and can also be used for small electronic devices having an imaging function, such as smartphones, tablet terminals, laptop computers, and the like.

What is claimed is:

1. An imaging apparatus comprising:
an image sensor;
a lens system configured to form an image of an object on an imaging surface of the image sensor; and
a branching optical system configured such that an image-forming light flux in the lens system is split and propagated into a plurality of optical paths having different optical path lengths and such that the principal rays of a plurality of image-forming light fluxes obtained after the splitting are incident on a predetermined region of the image sensor.

2. The imaging apparatus according to claim 1, wherein
the branching optical system includes:
an optical path branching surface that splits the image-forming light flux before the splitting into a first optical path and a second optical path; and
at least one reflective surface arranged such that light through the first optical path and light through the second optical path are incident on the image sensor.

3. The imaging apparatus according to claim 1, wherein the branching optical system includes a Michelson interferometer type optical system that includes a beam splitter and two mirrors.

4. The imaging apparatus according to claim 3, wherein
the beam splitter is a polarization beam splitter, and
the imaging apparatus further comprises a quarter-wave plate provided between each of the two mirrors and the beam splitter.

5. The imaging apparatus according to claim 3, wherein the branching optical system further includes at least one parallel plate inserted between at least one of the two mirrors and the beam splitter, and the parallel plate is configured to partially reflect light at the front and back surfaces thereof.

6. The imaging apparatus according to claim 1, wherein
the branching optical system includes:
at least one parallel plate provided on the object side of the lens system or on the image sensor side of the lens system, the parallel plate being configured to partially reflect light at the front and back surfaces thereof.

7. The imaging apparatus according to claim 1, further comprising:
an image processing unit configured to generate an image using an integrated point spread function of the lens system and the branching optical system.

8. An imaging apparatus comprising:
an image sensor;
a lens system configured to form an image of an object on an imaging surface of the image sensor; and
a branching optical system configured such that an image-forming light flux in the lens system is split and propagated into a plurality of optical paths and such that image-forming points of respective imaging-forming light fluxes after the splitting are located at different distances from the imaging surface.

9. The imaging apparatus according to claim 8, wherein
the branching optical system includes:
an optical path branching surface that splits the image-forming light flux before the splitting into a first optical path and a second optical path; and
at least one reflective surface arranged such that light through the first optical path and light through the second optical path are incident on the image sensor.

10. The imaging apparatus according to claim 8, wherein the branching optical system includes a Michelson interferometer type optical system that includes a beam splitter and two mirrors.

11. The imaging apparatus according to claim 10, wherein
the beam splitter is a polarization beam splitter, and
the imaging apparatus further comprises a quarter-wave plate provided between each of the two mirrors and the beam splitter.

12. The imaging apparatus according to claim 10, wherein the branching optical system further includes at least one parallel plate inserted between at least one of the two mirrors and the beam splitter, and the parallel plate is configured to partially reflect light at the front and back surfaces thereof.

13. The imaging apparatus according to claim 8, wherein
the branching optical system includes:
at least one parallel plate provided on the object side of the lens system or on the image sensor side of the lens system, the parallel plate being configured to partially reflect light at the front and back surfaces thereof.

14. The imaging apparatus according to claim 8, further comprising:
an image processing unit configured to generate an image using an integrated point spread function of the lens system and the branching optical system.

15. An imaging apparatus comprising:
an image sensor;
a lens system configured to form an image of an object on an imaging surface of the image sensor; and
a Michelson interferometer type optical system that is provided between the lens system and the image sensor and that splits light from the lens system into two arms having different optical path lengths and supplies resulting beams of light to the image sensor in an overlapped manner.

16. The imaging apparatus according to claim 15, further comprising:
an image processing unit configured to generate an image using an integrated point spread function of the lens system and the branching optical system.

17. An imaging apparatus comprising:
an image sensor;
a lens system configured to form an image of an object on an imaging surface of the image sensor; and
an optical system that includes at least one parallel plate provided on the object side of the lens system or on the image sensor side of the lens system, the parallel plate being configured to partially reflect light at the front and back surfaces thereof.

18. An endoscope comprising the imaging apparatus according to claim 1.

19. An endoscope comprising the imaging apparatus according to claim 8.
